(19) United States
(12) Patent Application Publication
(10) Pub. No.: US 2008/0195322 A1
(43) Pub. Date: Aug. 14, 2008

(54) Quantification of the Effects of Perturbations on Biological Samples

(75) Inventors: Steven J. Altschuler, Dallas, TX (US); Lit-Hsin Loo, Dallas, TX (US); Lani F. Wu, Dallas, TX (US)

Correspondence Address: FULBRIGHT & JAWORSKI L.L.P., 600 CONGRESS AVE., SUITE 2400, AUSTIN, TX, 78701, US

(73) Assignee: The Board of Regents of the University of Texas System

(21) Appl. No.: 11/673,910

(22) Filed: Feb. 12, 2007

(51) Int. Cl. G01N 33/48

(52) U.S. Cl. 702/19

(57) ABSTRACT

A multivariate, automated and scalable method for extracting profiles from images to quantify the effects of perturbations on biological samples. Morphological features are determined from images of treated (perturbed) and control (unperturbed) biological samples, and multivariate classification, for example, using a separating decision hyperplane, is used to separate the distribution of measured feature data into control and treated groups. This classification may be used to determine a magnitude of the effect of the particular perturbation under study. A practical application is high-throughput image-based drug screening, wherein the effects of many different compounds, each applied at different doses and for different exposure times, may be profiled to, for example, characterize compound activities and to identify dose-dependent multiphasic drug responses, or to determine and classify the biological effects of new compounds.

### BACKGROUND

The recent increased availability of high-precision robotic liquid handling machinery, automated imaging techniques, and high-performance computing has enabled advances in the development of high-throughput image-based biological assays. These assays enable the quantitative observation of cellular phenotypes, including morphological changes, protein expression, localization, and post-translational modifications, from biological samples, such as single cells. Automated image processing algorithms for cell segmentation and feature extraction offer the ability to extract objective measurements of these multidimensional phenotypes, and are particularly useful for the analysis of image data sets that are too large, or of phenotypes that are too subtle, for reliable human scoring. Comparisons of these measurements obtained from biological samples in different experimental conditions may be used to derive profiles that summarize phenotypic changes in response to different pharmacological or physiological perturbations, and presumably reveal important biological effects. Several recent studies have developed high-throughput image-based assays approaches to build profiles to characterize drug effects, screen for small molecules, classify sub cellular localizations, and characterize whole-genome phenotypes by using RNA interference or gene-deletion libraries.

In addition, quantitative measurement of a drug effect on biological samples is an important step toward discovering new drug candidates. To accomplish this, quantitative measurements of phenotypes, also referred to as features, are made on biological samples treated with a drug of interest. A profile, which characterizes the phenotypic changes between the treated and untreated biological samples, is then derived from features collected from these biological samples. Ideally, drugs with similar targets should have similar profiles; while drugs with dissimilar targets should have dissimilar profiles.

Profiling methods based on genomic, proteomic, or metabonomic assays have been used to study drug effects. However, these methods usually work on DNA or protein collected from cell lysate, and therefore fail to capture changes at the single cell level. When profiling at the individual cell level is required, flow cytometry may be used to identify subpopulations of cells with similar profiles. One of the disadvantages of flow cytometry is that features containing morphology and spatial information, such as sub cellular localization of a protein, co-localization of proteins and shape of a sub cellular organelle, are not measured.

Fluorescence microscopy, which is capable of extracting a richer set of features than flow cytometry, provides an alternative for building drug profiles at the single cell level. In fluorescence microscopy, proteins or organelles of interest inside a cell are labeled with fluorescence markers, which emit light when excited. Then, a variety of morphology- and intensity-based features, such as the total intensity, the area, and the eccentricity of each measured fluorescent region, may be extracted from such a fluorescence microscopy image.

However, several bottlenecks in data analysis have limited the full potential of high-throughput image-based assays. First, one of the challenges has been to effectively transform distributions of multivariate, phenotypic measurements from single cells into multivariate profiles that are both machine and human interpretable. Common univariate profiling approaches miss feature correlations at the single-cell level. Second, beyond the standard challenges of image preprocessing, cell segmentation, and feature extraction, which are partially solved by available automated image analysis software, it is in fact not apparent which or how many features should be measured. An unbiased approach allowing for the discovery of unexpected phenotypes calls for the inclusion of many objective measurements. However, the inclusion of irrelevant features not only increases the overhead of computation and storage, but also reduces the sensitivity of the data analysis. A final challenge has been to determine the effective dosage ranges and quantify possible dose-dependent multiphasic response of a compound. Traditional dose-response curves based on viable cell counts fail to distinguish between different responses of a compound within effective concentrations. This step is essential for discovering novel mechanisms of known compounds.

Thus, although these prior profiling methods attempted to build multidimensional profiles of cells by extracting a large number of features from microscopy images, the profiling methods proposed by them suffer from one or more of the following shortcomings:

Univariate—Each extracted feature was treated independently and profiles were not built from all features simultaneously. It should be noted that profiles built from multivariate features, such as the ratio of two features or the projections of multiple features into principal components, are not fully multivariate if the profiles are computed by only considering proper subset of the features.

Non-automated—Profiles were not built and compared automatically. Manual visual grouping of data points was used.

Poorly scalable—Each drug profile was built by using information extracted from the feature values of all the drugs considered. Thus, the addition of a new drug requires the recalculation of all profiles. As the number of drugs becomes large (>10,000), these methods may become computationally prohibitive. Examples for these methods include principal component projection and supervised classification. It would be preferable to extract a drug profile independent of other drug profiles.

This listing failings of prior approaches is not considered to be exhaustive, and other failings will also be apparent to one of ordinary skill in this field.

### SUMMARY

Presented is a compound profiling method that is multivariate, automated and scalable. The method takes into consideration all features simultaneously. Thus, it can produce profiles that give better separation of compounds, such as drugs, with different targets and association of compounds with similar targets than existing univariate approaches. The multivariate profiling approach of the present disclosure considers dependencies among features, and improves the ability to characterize, compare, and predict cellular changes in response to external perturbations.

One aspect of the invention is a method of profiling the effects of perturbations on biological samples, including, imaging control biological samples and perturbed biological samples to produce respective biological sample feature distributions in a multidimensional feature space, separating the control biological sample feature distribution and perturbed biological sample feature distributions using multivariate classification, and profiling the biological cell perturbations based on the separations.

Imaging may be, for example, by fluorescence microscopy, brightfield microscopy, differential interference contrast microscopy, phase contrast microscopy, confocal microscopy, flow cytometry, or any other acceptable imaging method. The biological samples may include, for example, cells, tissues, biopsies or serum samples. The perturbations may be, for example, pharmacological (for instance, drugs, chemical compounds, toxins, and/or synthetic or natural products), physiological (for instance, insulin, hormones, steroids, and/or peptides), environmental (for instance, temperature, radiation and/or pressure), or genetic perturbations (for instance, microRNA, siRNA, mutation, mutagenesis (chemical, transposition, radiation) and/or genetic insertions and/or deletions). Usable multivariate classification algorithms used may be, for example, a support vector machine that produces separating hyperplanes and classification accuracies, neural networks or classification and regression tree (CART) algorithms, among others.

An optional aspect of the invention includes reducing the feature set by selectively removing features from the feature distributions, reapplying multivariate classification after the selected features have been removed, and repeating the selective removal and reapplying steps until a classification accuracy is below a predetermined minimum.

Yet another aspect of the invention is a compound screening method, including, treating biological samples with a plurality of compounds, for example drugs, each at a plurality of concentrations, to produce treated biological samples, imaging an untreated biological sample and the treated biological sample to produce untreated and treated biological sample feature distributions in a multidimensional feature space. Then, multivariate classification is applied to the untreated and treated biological sample feature distributions using, for example a support vector machine algorithm to determine separating hyperplanes. Finally, the compounds are screened based on multivariate profiles derived from the separating hyperplanes.

Another aspect is titration clustering which may be performed on the multivariate profiles derived from the multivariate classification algorithm based on the plurality of concentrations of the compounds. Titration clustering may be used to determine biologically effective compound dosages and separating compound dosages with different biological effects.

The method may be used to screen compounds to determine efficacy for treating a target condition, or to determine common effects of different compounds.

The terms “a” and “an” are defined as one or more unless this disclosure explicitly requires otherwise.

The terms “substantially,” “about,” and “approximately,” their variations are defined as being largely but not necessarily wholly what is specified as understood by one of ordinary skill in the art, and in one non-limiting embodiment, the substantially refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Other features and associated advantages will become apparent with reference to the following detailed description of specific embodiments in connection with the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a flowchart of an embodiment of the present invention.

FIG. 2 is a separating hyperplane in accordance with aspects of the present invention.

FIG. 3 is a flowchart of dosage range profile determination in accordance with aspects of the present invention.

FIGS. 4A and 4B are dendograms illustrating aspects of the present invention.

FIGS. 5A and 5B are tables showing stock plate layout in accordance with aspects of the present invention.

FIG. 6 is a compound list used to illustrate aspects of the present invention.

FIG. 7 is a cell feature list in accordance with aspects of the present invention.

FIGS. 8A-D are graphs illustrating multiphasic compound effects in accordance with aspects of the present invention.

FIG. 9 is a table illustrating drug screening performance in accordance with aspects of the present invention.

FIG. 10 is another dendogram illustrating aspects of the invention.

FIGS. 11A-D are graphs illustrating compound category prediction in accordance with aspects of the present invention.

### DETAILED DESCRIPTION

The invention and the various features and advantageous details are explained more fully with reference to the nonlimiting embodiments that are illustrated in the accompanying drawings and detailed in the following description. Descriptions of well known starting materials, processing techniques, components, and equipment are omitted so as not to unnecessarily obscure the invention in detail. It should be understood, however, that the detailed description and the specific examples, while indicating embodiments of the invention, are given by way of illustration only and not by way of limitation. Various substitutions, modifications, additions, and/or rearrangements within the spirit and/or scope of the underlying inventive concept will become apparent to those skilled in the art from this disclosure.

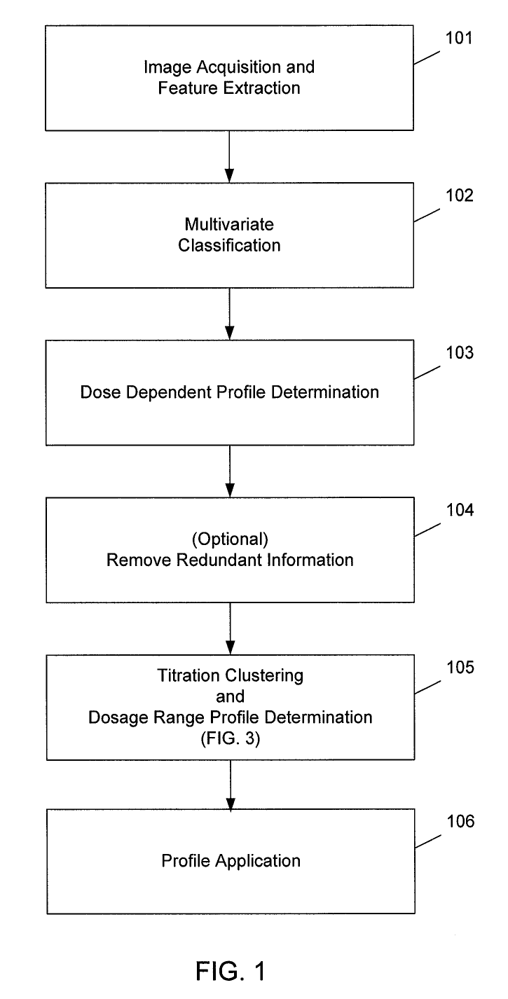

Referring to FIG. 1, presented is a flowchart of an embodiment of the present disclosure. Beginning in step 101, low-level image preprocessing, cell segmentation and image feature extraction algorithms are applied to images of treated and control biological samples.

The biological samples may be, for example, individual cell populations, tissues, biopsies or serum samples, and the treatment or perturbation of the biological samples may take many forms including, for example, pharmacological (for instance, drugs, chemical compounds, toxins, and/or synthetic or natural products), physiological (for instance, insulin, hormones, steroids, and/or peptides), environmental (for instance, temperature, radiation and/or pressure), or genetic perturbations (for instance, microRNA, siRNA, mutation, mutagenesis (chemical, transposition, radiation) and/or genetic insertions and/or deletions). The images may be obtained using various known techniques, including, for example, fluorescence microscopy, brightfield microscopy, differential interference contrast microscopy, phase contrast microscopy, confocal microscopy, flow cytometry, or any other acceptable imaging method.

The phenotype of each cell is represented by a vector of measured values in the multidimensional feature space. The phenotypes of the populations of treated and control cells are thereby represented as two distributions of points within the multidimensional feature space. These two distributions may be highly overlapping at low compound dosages, while easily separable at high compound dosages. For imagining, biological samples may be exposed to a serial compound titration and to a control condition, and may be fixed, stained with fluorescent markers if appropriate for the imaging technique employed, and imaged. If appropriate for the particular application, automated cell segmentation software identifies the DNA and cell boundaries. Image processing tools may quantify properties (such as intensities, textures, and morphologies) of the fluorescent markers, and may represent each cell in the biological sample as points in a high-dimensional feature space.

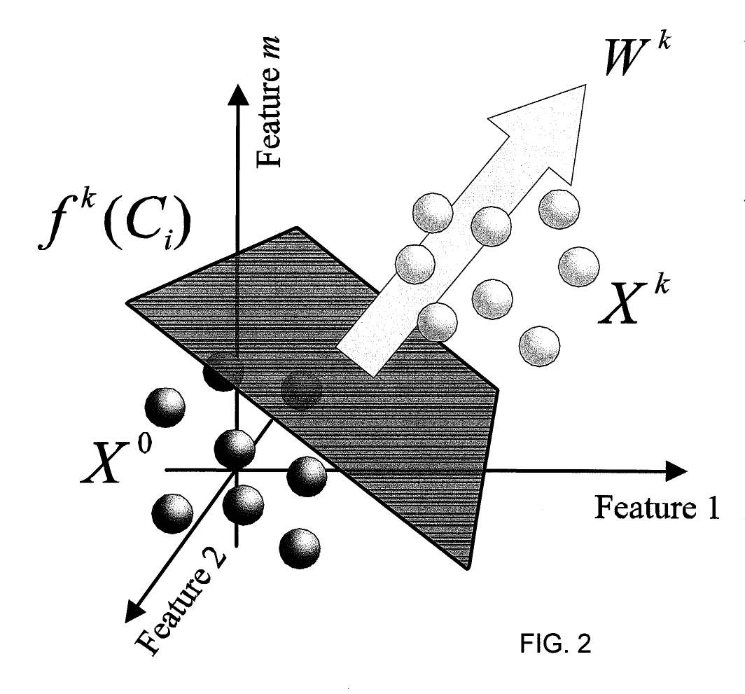

In step 102, for each dosage, a multivariate classification algorithm is applied to classify imaged biological samples into treated and untreated classes for each compound concentration. The multivariate classification algorithm, may be, for example, a support vector machine that produces separating hyperplanes and classification accuracies, neural networks or classification and regression tree (CART) algorithms, among others. When a separating hyperplane is used to classify the imaged biological samples into treated and untreated classes, the hyperplane may be determined, for example, using a support vector machine (SVM) algorithm which produces a separating hyperplane, a normal vector and a classification accuracy. The unit normal vector to the hyperplane is a multivariate measurement indicating the direction of maximum separation of the two distributions, and the coefficients of the unit normal vector indicate the relative importance of each feature in deciding whether a cell belongs to the treated or control class, as explained in more detail with reference to FIG. 2.

In step 103, a dose-dependent profile is determined from the multivariate classification determined in step 102. Since a single compound at different dosages, and different compounds with different targets, may induce different phenotypic changes, when hyperplanes are used, the normal vector of the separating hyperplane may be used as a multivariate compound-dosage profile. The classification accuracy of the hyperplane may be estimated using standard k-fold cross-validation. The classification accuracy of perfectly separated distributions is 100%, while the accuracy of a random classification is 50%. By classifying different sets of control biological samples from each other, an empirical null distribution of classification accuracy may be estimated, and a classification significance threshold of p=0.05 may be set. At each compound concentration index i, the weight vector Wᵢ of the hyperplane defines a profile of the compound at that concentration. The performance of Wᵢ is given by the classification accuracy of the hyperplane. A threshold for classification accuracy may be determined above which classifications are deemed significant. More details regarding profile determination are discussed below with reference to FIG. 2.

In optional step 104, for each extracted profile, redundant and non-informative features may be removed, using, for example, recursive feature removal with reclassification using the multivariate classification algorithm after feature removal. When employed with separating hyperplanes, this is an iterative process that removes feature dimensions corresponding to the coefficients of smallest absolute value in the profile vector, and then recomputes the separating hyperplane. The process of dimension reduction continues until the classification accuracy of the hyperplanes decreased significantly. The dimensionally reduced profiles may then be mapped back to the original feature space by padding with zeros in order to allow comparisons of profiles in the same dimension.

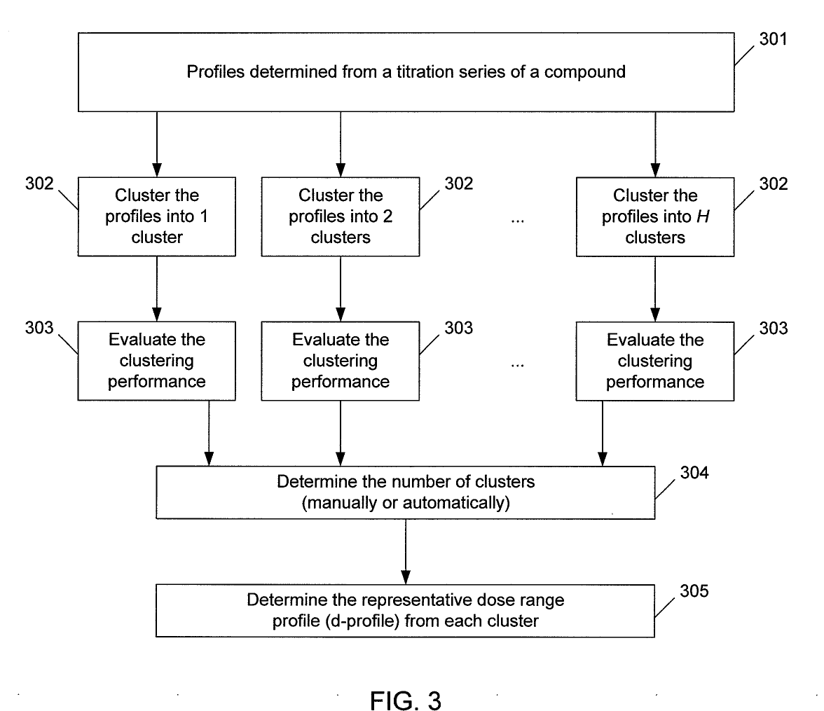

In step 105, a clustering algorithm is used to partition the titration series for each compound into ranges with maximum profile similarity, and a representative dosage range profile (d-profile) is determined from each of the determined titration ranges. Before the clustering, a reproducibility score indicating the similarity of dosage profiles across technical replicates is calculated and replicate profiles combined using vector averaging. The clustering may be performed on the combined profiles and the number of clusters may be determined automatically. For example, for each partition, a representative dosage range profile (d-profile) may be obtained by averaging the partition's constituent profiles that are both statistically significant and reproducible (as determined, for example, by a replicate reproducibility score threshold). Step 104 allows compounds to have multiple d-profiles across titrations, representing possible multiphasic responses. Clusters with no d-profiles may be discarded from further analysis, allowing the automated removal of low dosage ranges with no measured phenotypic effects and dosage ranges with poor replicate reproducibility. A compound may have more than one average d-profile, representing different effects at different concentrations. More details regarding dosage range profile determination are discussed below with reference to FIG. 3.

In step 106, multivariate profiles extracted from a library of compounds may be used in typical applications of high-throughput image-based assays, such as drug screening, phenotypic change detection, and category prediction. For drug screening, compounds with d-profiles most similar to that of a reference d-profile may be selected to be lead candidates. For phenotypic change detection informative features may be selected and compared for a subset of the profiles that gave the best drug screening performance. For category prediction, the category of an “uncharacterized” compound may be inferred from previously categorized compounds with similar d-profiles. In other words, profiles obtained from a library of compounds may be used for drug screening, phenotypic change discovery, and category prediction.

While drug screening is one example of a practical application of the present invention, other possible applications include: pathological applications such as tumor biopsies where reactions of non-transformed and transformed cells are compared to determine viability, drug resistance, and the like; molecular drug target/mechanism identification; and molecular pathway elucidation. Other applications are also contemplated.

If a support vector machine is used for multivariate classification (steps 101 and 102, FIG. 1), the details of the hyperplane determination include first, measuring m features in a known manner using an imaging technique such as fluorescence microscopy on nₖ cells in a biological sample that has been treated by perturbation (for example, drug) D^{k}, where k=0,1,2, . . . ,n_{D}. Here, the total number of perturbations considered in this example is n_{D}, and k=0 is reserved for values collected from the unperturbed (untreated) cells. The same m features are measured using fluorescence microscopy on nₖ cells that have been unperturbed (untreated) to provide a control corresponding to k=0. The value of the j-th feature measured on the i-th cell may be represented by a scalar x_{i,j}^{k}, where i=1,2, . . . ,nₖ, and j=1,2, . . . ,m; and all feature values obtained from the i-th cell may be represented by a row vector

Cᵢ^{k}=[x_{i,1}^{k}x_{i,2}^{k} . . . x_{i,m}^{k}]. Eq. 1

Cᵢ^{k} is a realization of a random vector C^{k}, which has a certain distribution in the m-dimensional feature space. For different D^{k}, the distribution of C^{k} will also be different. By performing the experiment, we obtained nₖ realizations of C^{k}, which may be combined into a data matrix

$${X}^{k} = [ \begin{matrix}{C}_{1}^{k} \\ {C}_{2}^{k} \\ ⋮ \\ {C}_{{n}_{k}}^{k}\end{matrix} ] = [ \begin{matrix}{x}_{1 , 1}^{k} & {x}_{1 , 2}^{k} & ⋯ & {x}_{1 , m}^{k} \\ {x}_{2 , 1}^{k} & {x}_{2 , 2}^{k} & ⋯ & {x}_{2 , m}^{k} \\ ⋮ & ⋮ & ⋰ & ⋮ \\ {x}_{{n}_{k} , 1}^{k} & {x}_{{n}_{k} , 2} & ⋯ & {x}_{{n}_{k} , m}^{k}\end{matrix} ] . \qquad Eq . 2$$

Given X^{k} and X⁰, where k≠0, the objective is to determine the profile of D^{k} under the experimental conditions. A profile is a row vector

W^{k}=[w₁^{k} w₂^{k} . . . w_{m′}^{k}], Eq. 3

which characterizes the difference between the distributions of C^{k} and C⁰. Note that m′, the dimension of W^{k}, may not be the same as m, the number of features.

If the measured features of the treated cells are similar to the untreated cells, i.e., no observable perturbation effect, the means of the distributions of C^{k} and C⁰ will be close to each other. If the perturbation induces observable feature changes on the cells, then the means of the distributions of C^{k} and C⁰ may be different from each other. This shift of distributions in the feature space may be characterized by a decision hyperplane that is optimally placed between the two distributions under a chosen criterion, which separates the two distributions.

For example, if there are two classes of cells: a negative class for the control (untreated) cells, and a positive class for the treated cells. The class label of a cell, Cᵢ^{k}, is denoted by yᵢ^{k}, where:

$${y}_{i}^{k} = \{ \begin{matrix}- 1 , & k = 0 \\ + 1 , & k \neq 0\end{matrix} \qquad Eq . 4$$

If Cᵢ represents a cell whose treatment is not known a priori. A decision function, f^{k} (Cᵢ), for D^{k} is a function that associates the cell, Cᵢ, with its class label by the following rule:

f^{k}(Cᵢ)≧0 [symbol] yᵢ=+1 Eq. 5

f^{k}(Cᵢ)<0 [symbol] yᵢ=−1

In this example, a linear decision function is used based on a hyperplane,

f^{k}(Cᵢ)= [symbol] W^{k},C [symbol] +b^{k}, Eq. 6

where <, > is the dot product operator in the Euclidean space Pm. This decision hyperplane is illustrated in FIG. 2, and is specified by W^{k} and b^{k}. The vector W^{k} is a vector normal to the hyperplane and the scalar b^{k} is a bias term.

Several possible methods of separation hyperplane determination may be used. For example, a support vector machine (SVM) algorithm may be used to select hyperplanes separating treated and untreated populations in the multidimensional feature space. Hyperplanes determined by this method provide both a unit normal vector, and a measure of classification accuracy. Alternatively, the hyperplanes may be chosen that give the minimum Bayes decision error or that maximize the distance between two classes while minimizing average distance within each class, or that maximizes its margin with respect to the two distributions, defined to be:

$${γ}^{k} = {min}_{i} {y}_{i}^{k} \{ 〈 {W}^{k} , {C}_{i}^{k} 〉 + {b}^{k} \} . \qquad Eq . 7$$

Other methods of selecting the appropriate hyperplane may also be acceptable.

In the context of this example, the margin of a hyperplane will be positive if the control and treated cells are separable (i.e., no misclassification). If the control and treated cells are not linearly separable, a soft margin, which tolerates misclassifications, may be used. In this example, the soft margin approach was used to find the maximal margin hyperplane due to its robustness to noisy data and outliers, although methods would also be acceptable. The maximal margin hyperplane may be determined from a support vector machine algorithm in a known manner.

Since W^{k} specifies the orientation of the maximal margin hyperplane, this normal vector will point in the direction in which the distribution of C^{k} is shifting away from the distribution of C⁰, FIG. 2. Besides its geometrical interpretation as a normal vector of a hyperplane, W^{k} may also be interpreted as a weight vector that specifies the relative importance of each feature in the decision function. Perturbations (for example, drugs) with different targets may induce changes in different features, and thus affect the importance of these features in deciding whether a cell has been treated or not. As a result, this weight vector wk may serve as a fingerprint for profiling a drug effect. In order to compare the weight vectors obtained from different perturbations, the weight vector may be optionally normalized so that its sum equal to 1.

One of the advantages of using W^{k} as a drug profile is that W^{k} is fully multivariate because the profiling method uses all features concurrently. Another advantage is that the building of W^{k} only requires X^{k} and X⁰, thus the complexity of the profiling algorithm is independent of n_{D}. This kind of profiling method is well-suited for building profiles for huge number of drugs.

Turning now to the details of the dosage range profile (d-profile) determination (step 105, FIG. 1, and FIG. 3), for a titration series of a compound (t=1,2, . . . ,T, where t is a titration index representing the concentration of the compound and T is the number of unique concentrations), a set of profiles {Wₜ^{k}} for the same drug D^{k} at different concentrations is determined as previously described, where t=1,2, . . . ,T, in step 301.

In step 302, given a maximum limit of the number of clusters, H, a clustering algorithm is used to cluster {Wₜ^{k}} into h clusters, for each h=1,2, . . . ,H. For example, a combinatorial clustering algorithm, which searches through all the possible partitions of {Wₜ^{k}} into h clusters for the optimum partition that minimizes a loss function, may be used. For example, the following within cluster point scatter can be used as a loss function.

$$L ( G ) = \frac{1}{2} {\sum }_{i = 1}^{h} {\sum }_{G ( t ) = i} {\sum }_{G ( {t}^{′} ) = i} d ( {W}_{t}^{k} , {W}_{{t}^{′}}^{k} ) \qquad Eq . 8$$

where G(t) is the cluster membership assignment to the profile Wₜ^{k}, and d(Wₜ^{k},Wₜ^{k}) is the similarity between two profiles, Wₜ^{k} and W_{t′}^{k}. The combinatorial clustering algorithm may be speeded up by putting certain constraints on the clustering. For example, the constraint that all profiles within a cluster must come from consecutive titrations can be used. Other suboptimal clustering algorithms can also be used in step 302.

In step 303, for each clustering result, the performance of the clustering is determined. For example, a consistency value for the clustering result after many trials of random disturbance can be used. When a dataset has a small number of profiles (e.g. 10-20), such as in the case of clustering of profiles obtained at different titrations, previous approaches based on resampling produces disturbances with low diversity. To overcome this difficulty, disturbance based on randomly generated, normally distributed noise can be used. The mean and the standard deviation of the noise were set to be zero and the standard deviation of the feature respectively. The algorithm is described below:

Given the number of cluster, h, and a set of profiles:
1. Add random noise to the original dataset to generate a training dataset.
2. Add random noise to the original dataset to generate a test dataset.
3. Cluster each of the training and test datasets into h clusters, and assign a cluster label (from 1 to h) to each profile.
4. Train a nearest-neighbor classifier on the training dataset.
5. Predict the cluster label of the test dataset using the trained classifier.
6. Calculate the consistency ratio by dividing the number of profiles with different predicted and assigned cluster memberships by the total number of profiles. Since the same cluster may have different predicted and assigned class labels, a matching algorithm, for example the Hungarian method, can be used to find the optimum matching between the two label sets.
7. Repeat steps 1-6 for 100 times, and calculate the average consistency ratio for h.
8. Repeat steps 1-6 for 100 times with a random classifier, and calculate the average random consistency ratio.
9. Normalize the average consistency ratio with the average random consistency ratio.

In step 304, the optimum number of partitions was determined manually or automatically by choosing the clustering result with the minimum average normalized consistency ratio.

In step 305, a representative d-profile is derived from each partition of profiles. For example, a d-profile may be obtained by averaging the partition's constituent profiles that are both statistically significant and reproducible (as determined, for example, by a replicate reproducibility score threshold).

### EXAMPLES

### Example 1

### Univariate/Multivariate Comparative Example

To illustrate that W^{k} may be used as a drug profile, W^{k} were clustered from 23 compounds with different known targets. Since W^{k} may characterize drug effects, W^{k}'s from compounds with similar targets will form a cluster, while W^{k}'s from compounds with different targets will form separate clusters.

The list of compounds used and their known major target is listed in Table I. The data that was used were obtained from HeLa (human cancer) cells. Only groups of compounds that have more than four members were chosen. Multiple replicates of some compounds (Nacodazole, Scriptaid, and Emetine) were provided from the original dataset. Ideally, profiles from the replicates of a drug are expected to be the closest to the profile of another replicate of the same drug. The concentrations of the compounds used are the effective concentrations that have been determined previously. Plates with DNA, anillin, and SC35 markers were used in this example. A segmentation algorithm was used to segment cells from the obtained images, and values for 29 features were measured for each cell. Feature values for around 2500-5000 cells per compound were obtained.

TABLE I
List of Compounds Tested

| Group | Compounds | Major Target |
|---|---|---|
| K | Alsterpaullone (K₁), Indirubin | Kinase; CDK |
| | Monoxime (K₂), Olomoucine (K₃), | |
| | Purvalanol A (K₄), Roscovitine (K₅) | |
| M | 105D (M₁), Colchicine (M₂), | Microtubule |
| | Epothilone B (M₃), Griseofulvin (M₄), | |
| | Monastrol (M₅), Nocodazole (M₆, M₇, | |
| | M₈), Podophyllotoxin (M₉), Taxol | |
| | (M₁₀), Vinblastine (M₁₁) | |
| H | Apicidin (H₁), Oxamflatin (H₂), | Histone |
| | Scriptaid (H₃, H₄), Trichostatin (H₅) | Deacetylase |
| P | Anisomycin (P₁), Cycloheximide (P₂), | Protein |
| | Didemnin B (P₃), Emetine (P₄, P₅) | Synthesis |
| | Puromycin (P₆) | |

For each compound, all the treated cells were split into 5 equal partitions. For every combination of four partitions, an equal number of cells were randomly selected from all the control cells, and a support vector machine (SVM) algorithm was used to determine the maximal margin hyperplane between the control and treated cells. The same process was repeated five times with different random splitting of partitions. The final decision hyperplane was an average of all the obtained hyperplanes.

Besides building the hyperplanes, an additional profile was built for each compound by using a prior art univariate method. This prior art method was based on z-scores derived from the Kolmogorov-Smimov (KS) statistics between the control and treated distributions of each feature. The clustering result obtained from the multivariate method was then compared with the result obtained from this prior art univariate method.

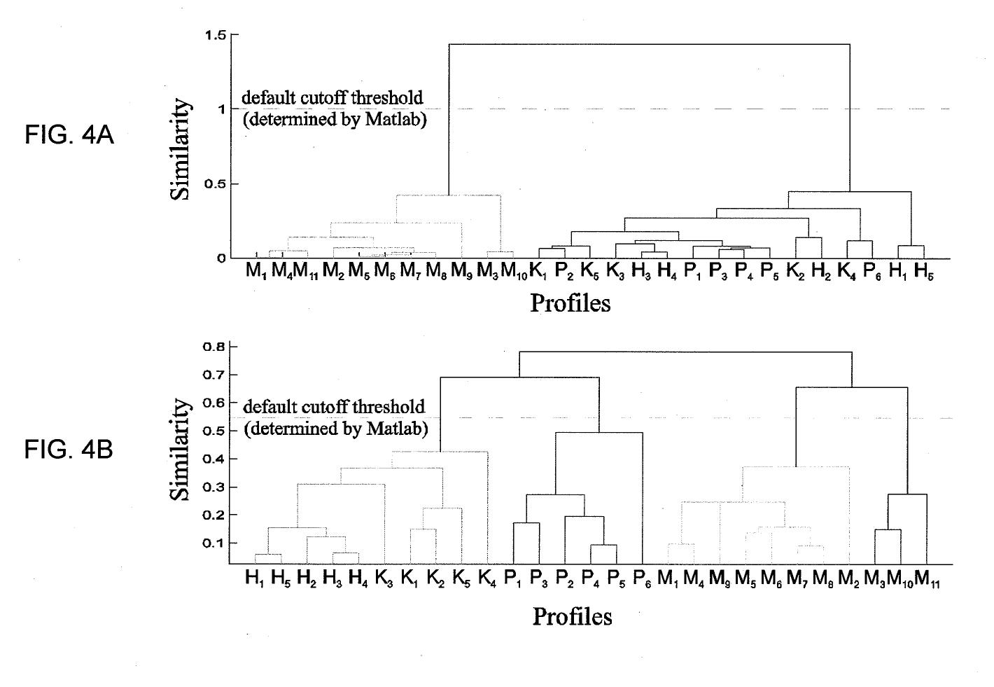

The profiles for all compounds were clustered by using a correlation-based hierarchical clustering algorithm, implemented in Matlab v14 SP3. The dendrogram obtained from the hierarchical clustering of the profiles obtained from the univariate profiling method is shown in FIG. 4A, and from the multivariate profiling method is shown in FIG. 4B. The vertical axis is the similarity between two connecting clusters; and the horizontal axis is the profile of a compound, which is labeled by the compound's group label as given in Table I. A default cutoff threshold, determined by Matlab's clustering algorithm, is also shown in each dendrogram in dashed line.

In the dendrogram of profiles obtained from univariate profiling, FIG. 4A, a cluster consisting of all compounds affecting microtubule (M) is formed. However, the profiles from all other compounds fail to be separated from each other; and replicate profiles of Nocodazole and Emetine are not consistently neighbors. In the dendrogram of profiles obtained from the multivariate profiling of the present disclosure, FIG. 4B, four clusters are automatically obtained. Two of the clusters consist of only compounds affecting microtubules (M), but they are linked together. Another cluster consists of only protein synthesis inhibitors (P). Although the last cluster consists of both CDK inhibitors (K) and histone deacetylase inhibitors (H), all histone deacetylase inhibitors forms a tight subcluster. Furthermore, only replicate profiles of Nocodazole are not consistently neighbors. The replicate profiles of Emetine were grouped together. Overall, the multivariate profiling of the present disclosure is able to group compounds according to their targets and gives better grouping than the univariate profiling. The clustering results show that W^{k}'s may be used as drug profiles.

### Example 2

### Comprehensive Phenotypic Profiling of 100-Compound Compendium

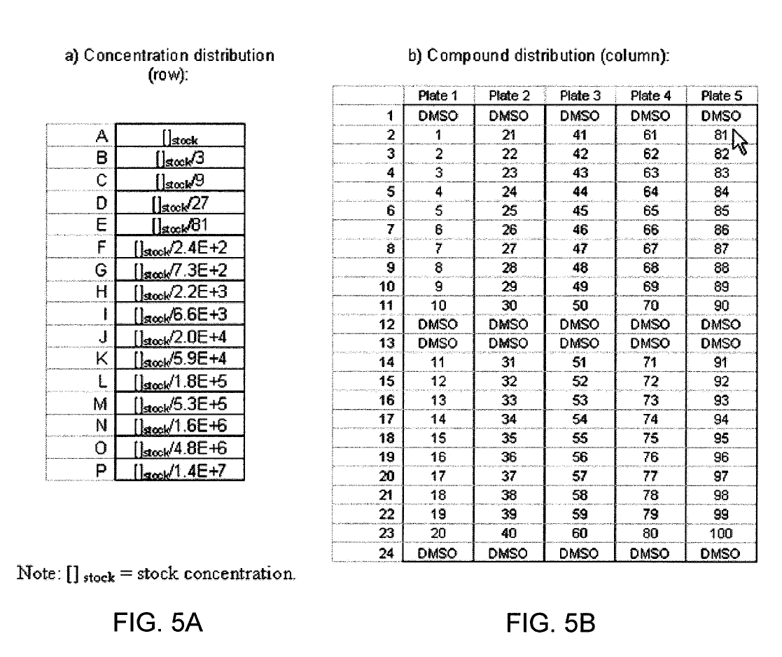

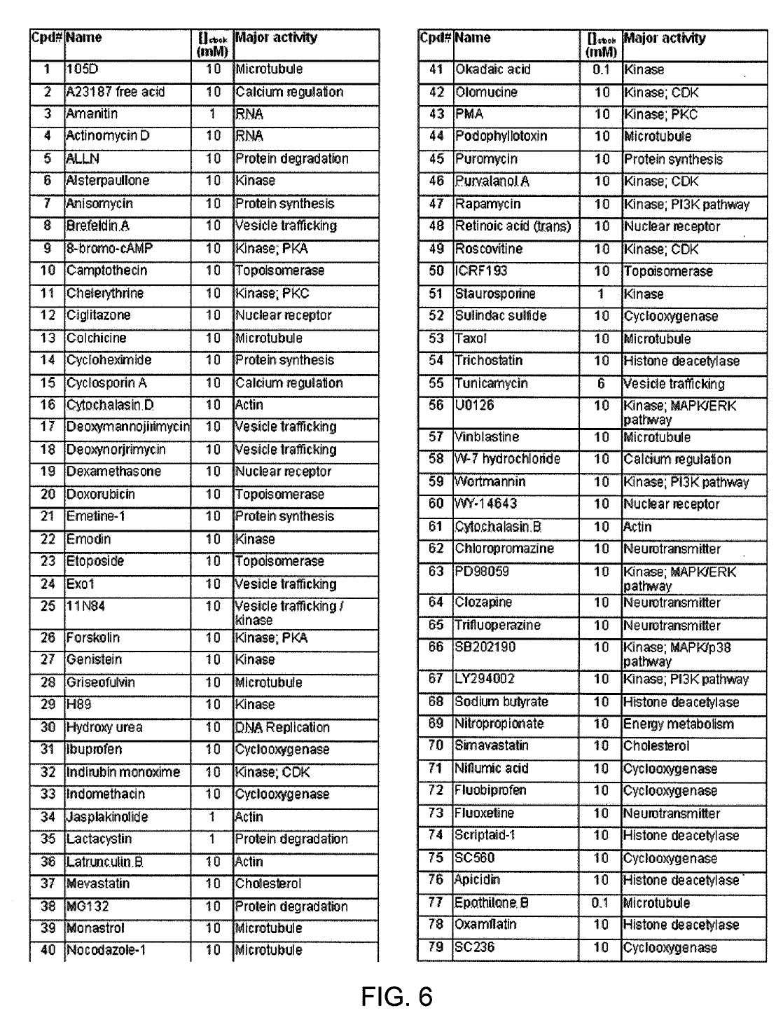

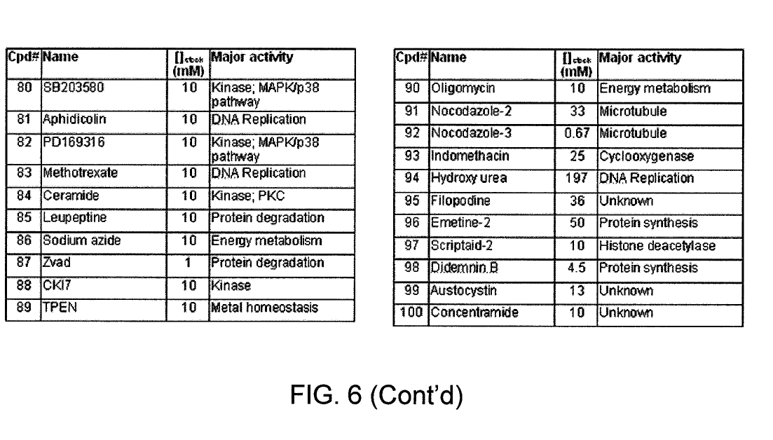

To illustrate the performance of the present multivariate approach, the disclosed methods were applied to a compendium of fluorescence microscopy images in which HeLa cells were treated with 100 compounds, dissolved in dimethyl sulfoxide (DMSO), over 13 threefold titrations as shown in FIGS. 5A and 5B. The compounds represented approximately 20 categories of activities as shown in the table of FIG. 6, selected to cover mechanisms of toxicity, signaling pathways, and therapeutic targets in cancer and other diseases. Compound effects were assayed in duplicate on 384-well plates, using four sets of multiplexed molecular markers (DNA-SC35-anillin; DNA-p53-cFos; DNA-p38-pERK; DNA-mirotubule-actin). On average, 2413±852 (mean±standard deviation) cells were captured per well, from 103,580 images per marker set, to yield a total of ˜37 million individual identified cells. Cells treated with DMSO alone were used as controls.

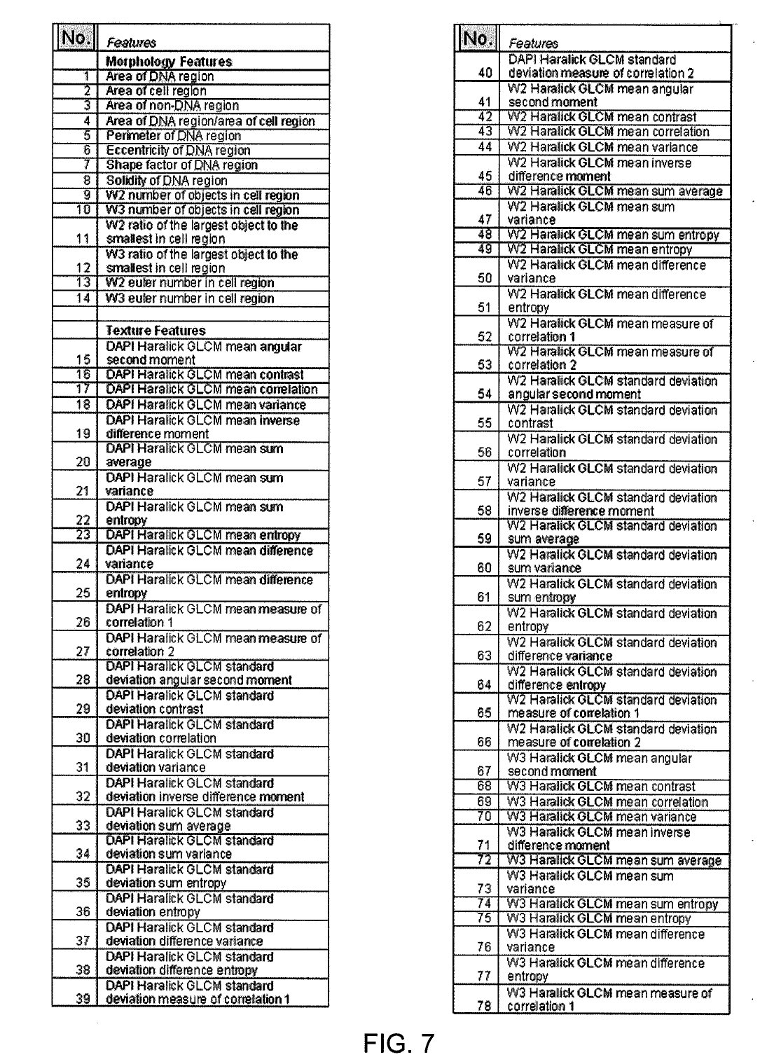

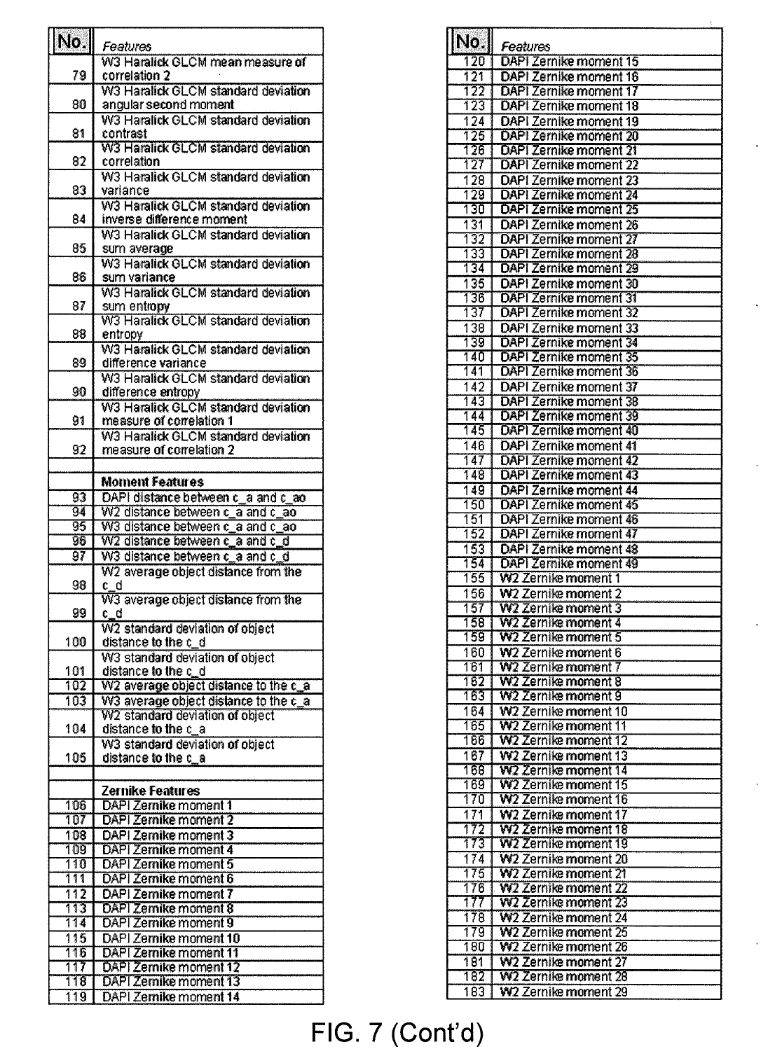

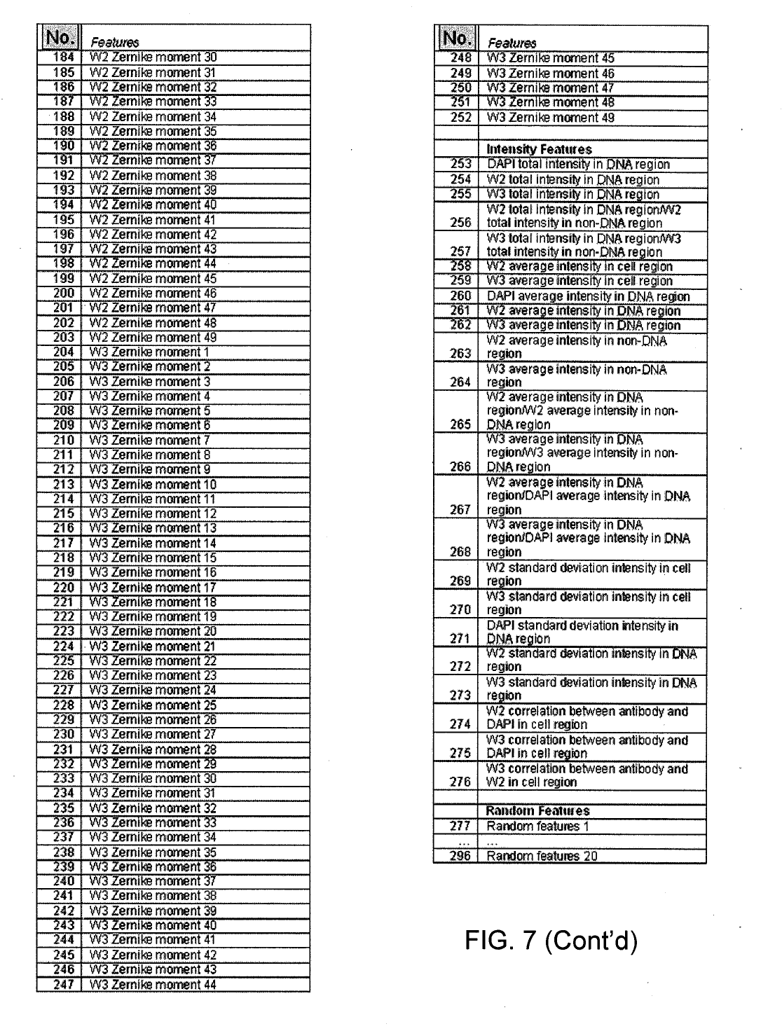

In order to gather a comprehensive collection of phenotypic measurements, for each marker set and each cell, the values of 296 image features were computed from the DNA and non-DNA regions as shown in FIG. 7, including 14 morphology features (measuring shape properties of the nuclear and cellular domains), 24 intensity features (measuring the expression levels of the stained proteins in different cellular compartments), 78 Haralick texture features (measuring the spatial patterns of stained proteins), 13 moment features and 147 Zernike features (both measuring the mass distributions of stained proteins). Although most of these features were derived from the measurements of individual markers, some features measured information from more than one marker (such as the spatial correlation between the intensities of two different markers). To demonstrate the robustness of the method in removing irrelevant features, 20 features with randomly generated values were also included.

For most of the compounds, the recursive feature removal step (optional step 104, FIG. 1) reduced the number of retained features needed for the optimum classification of the treated and control cells to around 20-40 features, indicating the original feature set was highly redundant for any particular compound. The random features that were intentionally generated were consistently eliminated early in the iterative process thus demonstrating the effectiveness of the ability to automatically remove features with little discriminative information. Among all the tested compounds, doxorubixin stood out to be the only compound whose effects could be detected by only a single feature in each of the four marker sets.

The importance of all feature categories were compared across different compounds on the same marker set. Despite the consistency in the number of retained features, the types of retained features were highly diverse. For example, on the DNA-SC35-anillin marker set, texture features were more important for Cholesterol inhibitors, but less important for compounds such as actin and DNA replication inhibitors. Overall, profile coefficients corresponding to texture and intensity features had the highest absolute values, while Zernike and moment features had comparatively lower absolute values.

Next, the importance of all feature categories were compared across different marker sets on the same compound. In general, texture features were more important than intensity features on the DNA-SC35-anillin and DNA-MT-actin marker sets; while the reverse was true on the DNA-cFos-p53 and DNA-p38-pERK marker sets. The results suggested that spatial pattern information was most relevant on the markers measuring cytoskeleton (DNA-MT-actin) or proteins with cell-cycle-dependent localization (DNA-SC35-anillin), while intensity information was most relevant on the markers measuring transcription factors (DNA-cFos-p53) or cell signaling proteins (DNA-p38-pERK).

### d-Profille Extraction

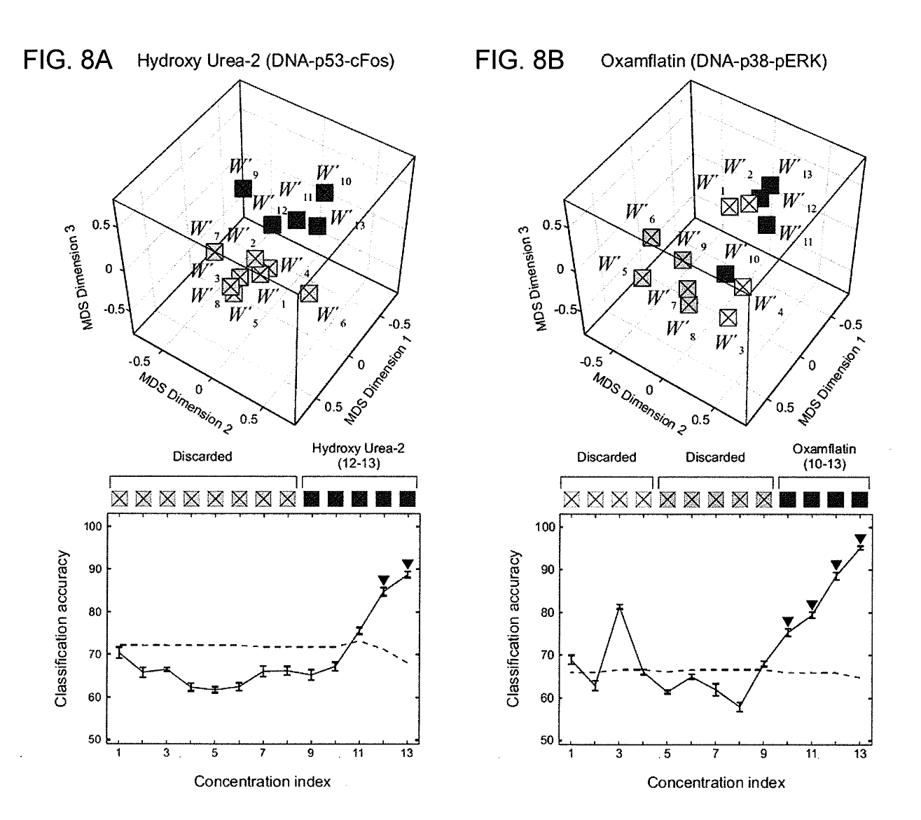

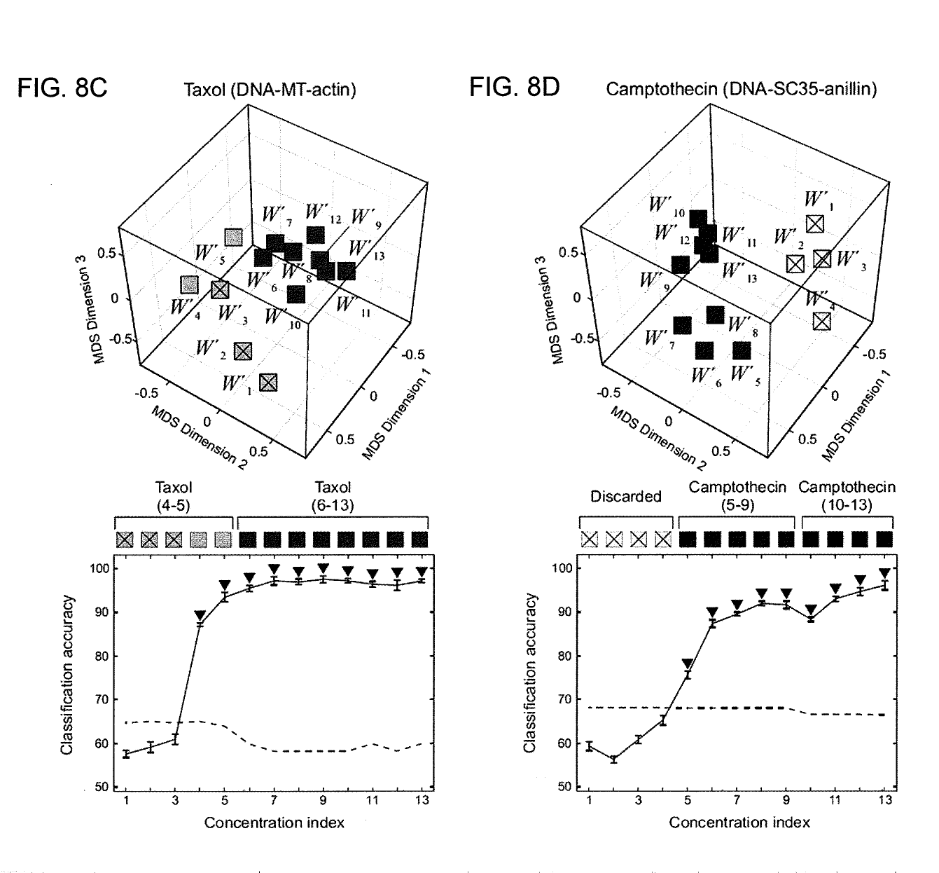

In this example, compound effects were considered significant only when the ability to separate treated from control cells was significantly greater than the ability to separate control cells from different wells. Due to biological and experimental variability, the significance thresholds of classification accuracy at p=0.05 estimated on every plate were much higher than 50% (FIGS. 8A-D, lower panels, dashed lines), and occasionally could reach as high as 90%. Therefore, well-to-well variability, even within the control population, could be high and should not be ignored.

The classification accuracy curves of most compounds showed classical sigmoidal dose-responses, with classification accuracies below the significance threshold at the lowest dosage ranges, and well above the significance threshold at the highest dosage ranges (FIGS. 8A-D, lower panels). For several compounds, classification accuracies trended slightly upwards for decreasing concentrations at the lowest dosages (FIGS. 8A-D, lower panels, concentration indices 1,2), likely due to microplate edge effects. Low dosages with significant classification accuracies were usually eliminated from computation of the final d-profiles as their reproducibility scores were mostly below threshold (FIG. 8B, lower panel, lack of triangle above concentration index 3).

The titration clustering algorithm (FIG. 1, step 105) yielded two clusters per compound on 65% of the compounds (FIGS. 8A, 8C, grouped boxes), and three clusters per compound on 35% of the compounds (FIGS. 8B, 8D, grouped boxes) over all four marker sets. The visualization of the inter-profile similarities using multi-dimensional scaling confirmed the existence of distinct clusters of profiles across the dosage-series of a compound (FIGS. 8A-D, upper panels). After removing profiles that were neither significant nor reproducible, one d-profile per compound was derived for 60% of the compounds (FIGS. 8A, 8B) and two or three d-profiles per compound were derived for 18% of the compounds (FIGS. 8C, 8D), corresponding to possible distinct dosage-dependent effects. The remaining 22% compounds did not give any d-profiles. In total, from the 100-compound compendium, 100, 100, 89, and 102 d-profiles were extracted using the DNA-SC35-anillin, DNA-p53-cFos, DNA-p38-pERK, and DNA-MT-actin marker sets respectively.

Across different marker sets, 73% of the compounds gave the same number of d-profiles on three or four marker set (p<0.01, permutation test), indicating significant consistency in the number of d-profiles extracted. For example, taxol consistently gave 2 d-profiles (FIG. 8C) across all four marker sets (concentration indices 4-5: 5 nM-16 nM, and concentration indices 7-13: 47 nM-35 μM).

### Drug Screening Performance

To simulate a drug screen for compounds of similar target to a known compound, a d-profile was selected to be the reference profile, while all other d-profiles from the compendium were used as blinded test profiles. Similarity scores between the reference profile and all other test profiles were computed and ranked. The test profiles that were most similar to the reference profile were selected as “drug candidates.”

For each reference profile, the performance in identifying test profiles was estimated with similar a target on each marker set by using prior target annotations as the “gold standard.” The receiver operating characteristic curve (AUC) was used as the performance evaluation criterion (Methods). “On-target” effects were defined as d-profiles whose AUC values were significant (p<0.05), and all other d-profiles were defined as “off-target.” 73%, 40%, 67%, and 56% of the compounds with more than one d-profile and at least one on-target d-profile had at least one off-target d-profile on the DNA-SC35-anillin, DNA-p53-cFos, DNA-p38-pERK and DNA-MT-actin marker sets respectively. For example, Camptothecin was found to have one on-target effect and one off-target effect. Thus, the present method can identify dose-dependent secondary or tertiary responses that were very different from the primary responses.

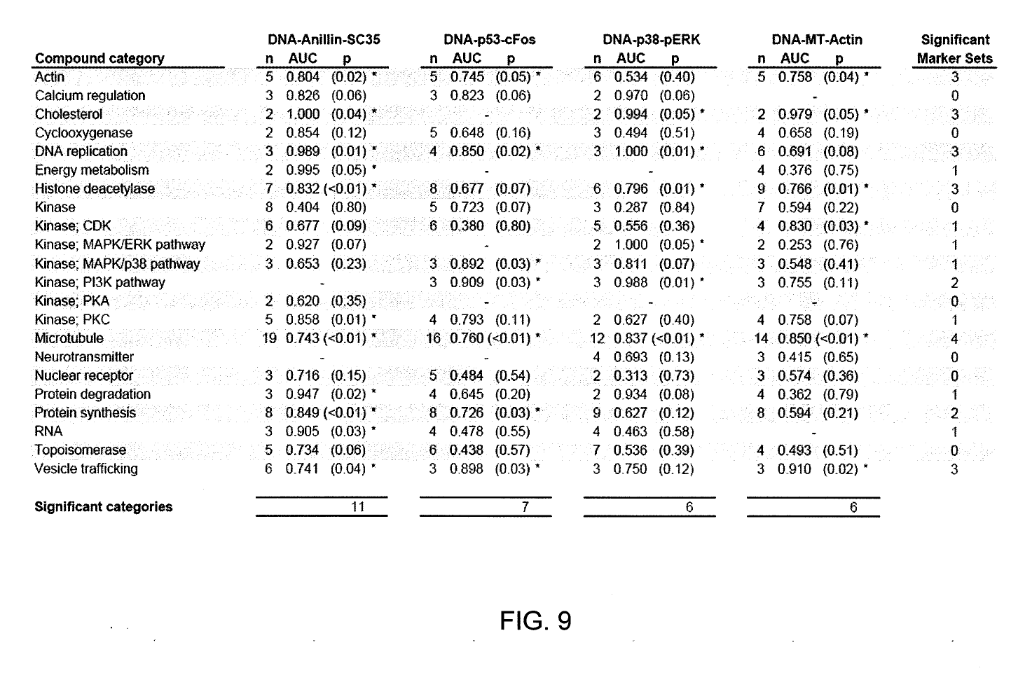

To summarize screening performance results, the AUC values of the compounds that had been annotated with the same target category were averaged for each marker set (FIG. 9, Table). Many compound categories gave statistically significant AUC values (48%, 30%, 26%, and 26% of categories in DNA-SC35-anillin, DNA-p53-cFos, DNA-p38-pERK, and DNA-MT-actin, respectively; FIG. 9), even though secondary or tertiary d-profiles were included in the averaging process. Three compound categories (cholesterol, DNA replication, and MAPK/ERK pathway inhibitors) gave perfect drug screening performance (AUC value=1).

The performance of a compound category across different marker sets were evaluated. Some compound categories induced phenotypic changes that were highly specific for the marker set used. For example, the effects of energy metabolism, PKC, protein degradation, and RNA inhibitors could only be detected by the DNA-anillin-SC35 marker set, while the effects of MAPK/ERK pathway inhibitors could only be detected by the DNA-p38-pERK marker set (FIG. 9). However, actin, cholesterol, DNA replication, histone deacetylase, microtubule, and vesicle trafficking inhibitors induced phenotypic changes that could be detected by using at least three of the marker sets (FIG. 9).

### Phenotypic Change Detection

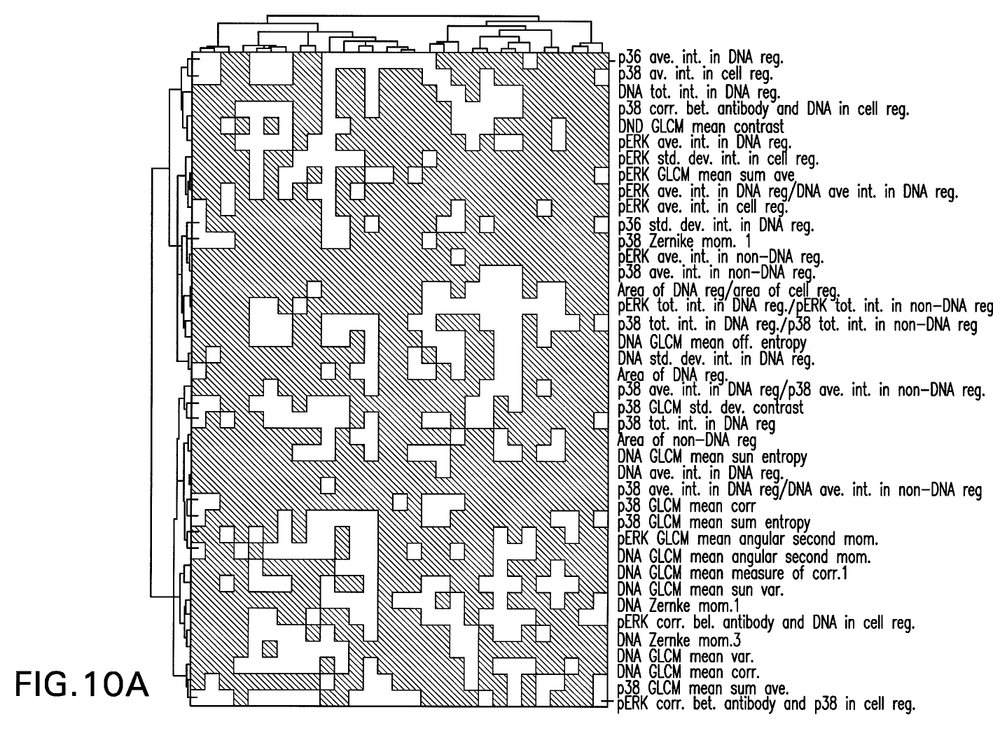

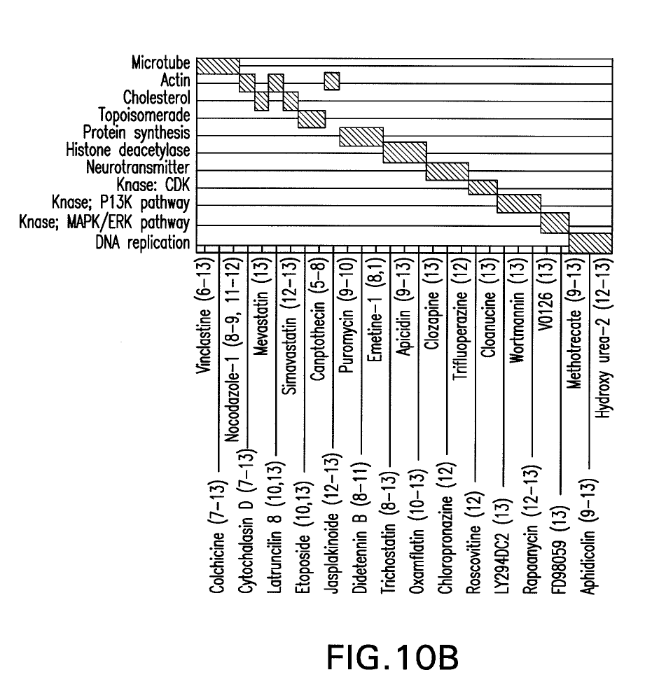

Another use of the method is to identify a small number of features that most discriminated compound categories. For each marker set and compound category, three representative on-target d-profiles were selected with maximum average AUC. The exclusion of off-target effects enabled the selection of on-target d-profiles from five compound categories not found significant in the drug screening process discussed above. Further, a hierarchical bi-clustering was performed on the 10-15 selected features from these d-profiles with the highest average absolute values on each marker set. A leaf-ordering algorithm was used to reorder the resulting dendrogram for the best visualization as shown in FIG. 10.

Since the most discriminative features from each compound category were used, near-perfect clustering of compounds by category was obtained. Some compounds were grouped together by obvious or easily interpretable phenotypic features, such as the area of DNA region and the ratio of p38 average intensity in DNA region over non-DNA region for compounds affecting DNA replication, while others were grouped together by non-obvious or novel phenotypic features, such as the DNA gray level co-occurrence matrix (GLCM) mean correlation and the p38 GLCM mean sum average for compounds annotated as neurotransmitter inhibitors. Some of these common phenotypic changes reflected cell cycle information, such as mitotic arrest, while some were independent of cell cycles, indicating that the present method provides more than cell cycle detection.

Further, the categories themselves formed natural “super-clusters” based on common blocks of features, which enabled the identification of common phenotypic changes among these categories. For instance, all the three categories of kinase inhibitors (CDK, PI3K and MAPK/ERK) formed a super-cluster sharing negative coefficients for the ratio of the pERK average intensity over the DNA average intensity in the DNA region, zero coefficient for the ratio of pERK total intensity in DNA region over the non-DNA region, and positive coefficient for the p38 average intensity in DNA region over the DNA average intensity in the DNA region.

### Category Prediction

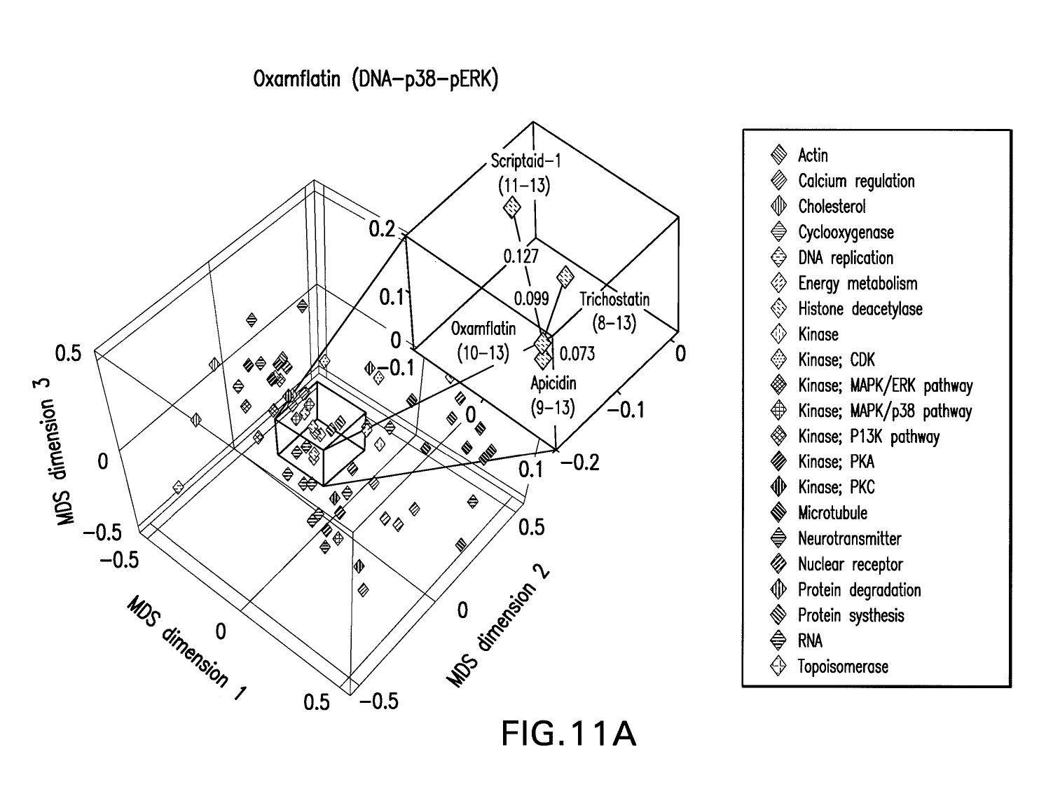

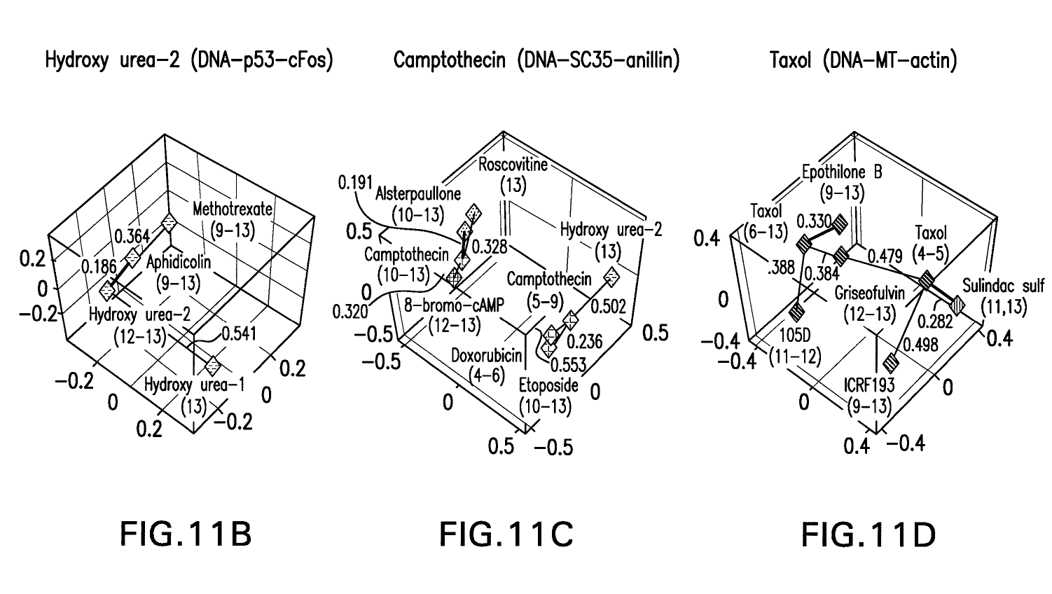

The compound category of a novel d-profile may be inferred by comparison to a collection of previously categorized reference d-profiles. For instance, comparison of d-profiles indicated that oxamflatin is most similar to trichostatin, scriptaid, and apicidin on the DNA-p38-pERK marker set (FIG. 11A). Although all of these compounds are histone deacetylase inhibitors, oxamflatin, trichostatin, and scriptaid are hydroxamic acids having very different chemical structures than apicidin, a cyclic tetrapeptide. Similarly for the DNA-p53-cFos marker set, the DNA replication inhibitor, hydroxy urea-2, was most found to be most similar to aphidicolin and methotrexate, both DNA replication inhibitors, as well as to a replicate of hydroxyl urea-2 with different starting stock concentration (FIG. 11B). To summarize the performance of category prediction, category prediction accuracies were estimated only on compounds with a single d-profile, since many compounds with multiple d-profiles had at least one off-target effect. For a d-profile, an accurate prediction was made when its most similar d-profile was annotated to the same target category.

Category prediction for compounds with multiple d-profiles was typically accurate for at least one of their d-profiles. For camptothecin, its first d-profile was closest to another topoisomerase inhibitor, etoposide, while its second d-profile was closest to a CDK inhibitor, alsterpullone (FIG. 11C). For taxol, its first d-profile was closest to sulindac sulfide, a cyclooxygenase inhibitor, while its second d-profile was closest to epothilone B and griseofulvin, which stabilize microtubule assemblies similarly to taxol despite dissimilarity in chemical structures (FIG. 11D). Microtubule depolymerizing compounds, such as 105D and nocadazale, were further away from this group of microtubule stabilizing compounds. These results indicate that the present method has the sensitivity to distinguish compounds affecting the same target, but through different mechanisms.

### CONCLUSION

From the above-described Example 2, it may be seen that the disclosed method of profiling compound-dosage responses reduces approximately 300 unbiased single-cell phenotypic features to approximately 20 maximally informative features for each marker set. The large reduction in dimensionality comes with greatly enhanced human interpretability of the drug response profiles and improved detection of novel cellular phenotypic changes, yet at little loss of classification accuracy. Analysis of these selected features demonstrated maximally informative marker and feature set combinations for detecting and discriminating among categories of compound classes, and will be applicable enable streamlining future drug screens.

According to the present disclosure, d-profiles effectively summarize high-throughput, single cell phenotypic responses to compounds. Separating compound dosage effects into multiple d-profiles results in more sensitive screening and raises the possibility of identifying novel dosage-dependent mechanisms, even for previously characterized compounds. The method of the present disclosure for building compounds is computationally and experimentally scalable; compound profiles are created independently of each other and allow for incremental growth of a compound compendium.

When applied to drug screening, the present method provides accurate quantification of complex phenotypic changes that are complementary to other high-throughput approaches, such as transcript profiling, and offers the potential to bring the use of model biological systems earlier into the drug discovery process. The method is also broadly applicable for characterizing single-cell phenotypic changes due to other external perturbations (such as, for example, cytokines, stress factors and RNA interference), and internal cellular states (such as, for example, diseased versus normal cells). It provides the basis for more sophisticated analysis, such as the characterization of synergistic or antagonistic behavior of combination of perturbations, identification of sub-populations of cells beyond commonly known states such as cell cycle, and reconstruction of biological pathways based on monitoring multi-dimensional phenotypic readouts.

All of the methods disclosed and claimed herein may be executed without undue experimentation in light of the present disclosure. While the methods of this disclosure may have been described in terms of preferred embodiments, it will be apparent to those of ordinary skill in the art that variations may be applied to the methods and in the steps or in the sequence of steps of the method described herein without departing from the concept, spirit and scope of the disclosure. All such similar substitutes and modifications apparent to those skilled in the art are deemed to be within the spirit, scope, and concept of the disclosure as defined by the appended claims.

1. A method of profiling the effect of a perturbation relative to the effect of another perturbation on biological samples, comprising:
subjecting each of at least first and second biological samples to a perturbation;
extracting multiple numerical features from the at least first and second biological samples after perturbation;
classifying the multiple numerical features extracted from each perturbed biological sample using a multivariate classification algorithm;
determining a multivariate profile of the effect a perturbation relative to the effect of another perturbation from the multivariate classification.

2. The method of claim 1, the perturbation including treatment with a compound at a concentration.

3. The method of claim 1, the perturbation including treatment with a mixture of compounds each at a concentration.

4. The method of claim 1, the perturbation including silencing the expression of a gene by RNA interference.

5. The method of claim 1, the perturbation including knocking out a gene.

6. The method of claim 1, the perturbation including treatment with a cytokine.

7. The method of claim 1, the perturbed cells including treatment with a free fatty acid.

8. The method of claim 1, the step of extracting multiple numerical features comprising:
labeling the at least first and second biological samples after perturbation using fluorescent probes to produce labeled cells;
illuminating the labeled biological samples using a light source;
imaging the labeled biological samples using fluorescence microscopy to produce biological sample images;
segmenting cell regions from the biological sample images; and
computing multiple numerical features from the segmented cell regions.

9. The method of claim 1, the step of extracting multiple numerical features comprising:
imaging the at least first and second biological samples after perturbation using brightfield microscopy to produce biological sample images;
segmenting cell regions from the biological sample images; and
computing multiple numerical features from the segmented cell regions.

10. The method of claim 1, the step of extracting multiple numerical features comprising:
imaging the at least first and second biological samples after perturbation using differential interference contrast microscopy to produce biological sample images;
segmenting cell regions from the biological sample images; and
computing multiple numerical features from the segmented cell regions.

11. The method of claim 1, the step of extracting multiple numerical features comprising:
imaging the at least first and second biological samples after perturbation using phase contrast microscopy to produce biological sample images;
segmenting cell regions from the biological sample images; and
computing multiple numerical features from the segmented cell regions.

12. The method of claim 1, the step of extracting multiple numerical features comprising:
labeling the at least first and second biological samples after perturbation using fluorescent probes to produce labeled cells;
passing the labeled cells through a flow cytometer;
illuminating the labeled cells using a light source;
detecting scattered and emitted light from labeled cells; and
computing multiple numerical features from the detected light.

13. The method of claim 1, the classifying and determining steps step comprising:
determining a separating hyperplane between the multiple numerical features extracted from each of the perturbed biological samples;
determining a normal vector and a classification accuracy score for each hyperplane; and
determining a multivariate profile of the effect of a perturbation relative to the effect of another perturbation from the normal vector.

14. The method of claim 13, the step of determining the separating hyperplane comprising, subjecting the features from the at least first and second biological samples after perturbation to a multivariate classification algorithm to determine the separating hyperplanes.

15. The method of claim 14, the classification algorithm comprising, a support vector machine algorithm.

16. The method of claim 13, the step of determining a multivariate profile from the normal vector comprising, dividing the normal vector with the sum of the absolute values of the elements of the normal vector.

17. The method of claim 1, further comprising, after the classifying step:
selectively removing features from the extracted multiple numerical features;
reclassifying multiple numerical features the after the selected features have been removed using a multivariate classification algorithm; and
repeating the selective removal and reclassifying steps until a classification accuracy classifying step is below a predetermined minimum to produce a reduced biological sample feature set.

18. The method of claim 13, further comprising:
after determining the classification accuracy score, comparing the classification accuracy score to a predetermined significance threshold; and
characterizing the perturbation as a function of the comparison.

19. A method of profiling the effect of a perturbation at a plurality of levels relative to the effect of a reference perturbation on biological samples, comprising:
subjecting a plurality of biological samples to a plurality of levels of a perturbation to produce a plurality of perturbed biological samples;
subjecting a biological sample to a reference perturbation to produce a reference perturbed biological sample;
extracting multiple numerical features from each of the perturbed biological samples;
classifying the multiple numerical features extracted from each perturbed biological sample using a multivariate classification algorithm; and
determining a plurality of multivariate profiles from the multivariate classification.

20. The method of claim 19, the step of subjecting to a plurality of levels of a perturbation comprising, treatment with a compound at a plurality of concentrations.

21. The method of claim 19, the step of subjecting to a plurality of levels of a perturbation comprising, treatment with a mixture of compounds at a plurality of mixing concentration ratios.

22. The method of claim 19, the step of subjecting to a plurality of levels of a perturbation comprising, treatment with a cytokine at a plurality of concentrations.

23. The method of claim 19, the step of subjecting to a plurality of levels of a perturbation comprising, treatment with a free fatty acid at a plurality of concentrations.

24. The method of claim 19, the step of extracting multiple numerical features, comprising:
labeling the perturbed biological samples using fluorescent probes to produce labeled biological samples;
illuminating the labeled biological samples using a light source;
imaging the labeled biological samples using fluorescence microscopy to produce biological sample images;
segmenting cell regions from the biological sample images; and
computing multiple numerical features from the segmented cell regions.

25. The method of claim 19, the step of extracting multiple numerical features, comprising:
imaging the perturbed biological samples using brightfield microscopy to produce biological sample images;
segmenting cell regions from the biological sample images; and
computing multiple numerical features from the segmented cell regions.

26. The method of claim 19, the step of extracting multiple numerical features, comprising:
imaging the perturbed biological samples using differential interference contrast microscopy to produce biological sample images;
segmenting cell regions from the biological sample images; and
computing multiple numerical features from the segmented cell regions.

27. The method of claim 19, the step of extracting multiple numerical features, comprising:
imaging the biological samples using phase contrast microscopy to produce biological sample images;
segmenting cell regions from the biological sample images; and
computing multiple numerical features from the segmented cell regions.

28. The method of claim 19, the step of extracting multiple numerical features, comprising:
labeling the perturbed biological samples using fluorescent probes to produce labeled biological samples;
passing the labeled biological samples through a flow cytometer;
illuminating the labeled biological samples using a light source;
detecting scattered and emitted lights from the illuminated labeled biological samples; and
computing multiple numerical features from the detected light.

29. The method of claim 19, the classifying and determining steps comprising:
determining a plurality of separating hyperplanes using the multivariate classification algorithm, each separating hyperplane being between the features extracted from a respective perturbed biological sample and features extracted from the reference perturbed biological sample;
determining a normal vector and classification accuracy score for each hyperplane; and
determining the plurality of multivariate profiles from the normal vectors.

30. The method of claim 29, the multivariate classification algorithm comprising, a support vector machine algorithm.

31. The method of claim 29, the step of determining the plurality of multivariate profiles from the normal vectors comprising, dividing the normal vectors with the sum of the absolute values of the elements of the normal vector.

32. The method of claim 19, further comprising, after the classifying step:
selectively removing features from the extracted multiple numerical features;
reclassifying the multiple numerical features using the multivariate classification algorithm after the selected features have been removed; and
repeating the selective removal and reclassifying steps until a classification accuracy of the classifying step is below a predetermined minimum to produce a reduced biological sample feature set.

33. The method of claim 29, further comprising:
after determining classification accuracy scores, comparing each classification accuracy score to a predetermined significance threshold; and
characterizing the respective perturbation as a function of the comparison.

34. The method of claim 19, further comprising:
after determination of the a plurality of multivariate profiles, performing titration clustering on the profiles.

35. The method of claim 34, further comprising:
after titration clustering, determining a representative profile from each cluster.

36. The method of claim 35, the step of determining a representative profile from each cluster, comprising:
determining profiles in a cluster that are not reproducible;
removing profiles in a cluster that are not reproducible; and
averaging the remaining profiles.

37. A method of profiling an effect on cells of a plurality of perturbations each at a plurality of levels relative to the effect of a reference perturbation, comprising:
subjecting a plurality of populations of cells to a plurality of perturbations each at a plurality of levels to produce a plurality of perturbed cell populations;
subjecting a population of cells to a reference perturbation to produce a reference perturbed cell population;
extracting multiple numerical features from each of the perturbed cell populations;
determining a plurality of separating hyperplanes, each being between the features extracted from a respective perturbed cell population and features extracted from the reference perturbed cell population;
determining a normal vector and classification accuracy score for each hyperplane; and
determining a plurality of multivariate profiles from the normal vectors.

38. The method of claim 37, the step of subjecting a plurality of populations of cells to a plurality of perturbations each at a plurality of levels comprising, treatment with a plurality of compounds each at a plurality of concentrations.

39. The method of claim 37, the step of subjecting a plurality of populations of cells to a plurality of perturbations each at a plurality of levels comprising, treatment with a plurality of compound mixtures each at a plurality of mixing concentration ratios.

40. The method of claim 37, the step of subjecting a plurality of populations of cells to a plurality of perturbations each at a plurality of levels comprising, silencing the expression of a plurality of genes by RNA interference.

41. The method of claim 37, the step of subjecting a plurality of populations of cells to a plurality of perturbations each at a plurality of levels comprising, knocking out a plurality of genes.

42. The method of claim 37, the step of subjecting a plurality of populations of cells to a plurality of perturbations each at a plurality of levels comprising, treatment with a cytokine at a plurality of concentrations.

43. The method of claim 37, the step of subjecting a plurality of populations of cells to a plurality of perturbations each at a plurality of levels comprising, treatment with a free fatty acid at a plurality of concentrations.

44. The method of claim 37, the step of determining the separating hyperplanes comprising, subjecting the features extracted from the respective perturbed cell populations and features extracted from the reference perturbed cell population to a multivariate classification algorithm to determine the separating hyperplanes.

45. The method of claim 44, the classification algorithm comprising, a support vector machine algorithm.

46. The method of claim 37, the step of determining a profile from the normal vector comprising, dividing the normal vector with the sum of the absolute values of the elements of the normal vector.

47. The method of claim 37, further comprising, after the step of determining a plurality of separating hyperplanes:
selectively removing features from the extracted features;
redetermining the separating hyperplanes after the selected features have been removed; and
repeating the selective removal and redetermining steps until a classification accuracy of the separating hyperplane is below a predetermined minimum to produce a reduced cell feature set.

48. The method of claim 37, further comprising:
after determining classification accuracy scores, comparing each classification accuracy score to a predetermined significance threshold; and
characterizing the respective perturbation as a function of the comparison.

49. The method of claim 37, further comprising:
after determination of the a plurality of profiles, performing titration clustering on the profiles.

50. The method of claim 49, further comprising:
after titration clustering, determining a representative profile from each cluster.

51. The method of claim 50, the step of determining a representative profile from each cluster, comprising:
determining profiles in a cluster that are not reproducible;
removing profiles in a cluster that are not reproducible; and
averaging the remaining profiles.

52. The method of claim 50, further comprising:
after the step of determining the representative profile, screening the perturbations to determine perturbations with representative profiles most similar to a target perturbation.

53. The method of claim 50, further comprising:
after the step of determining the representative profile, comparing the representative profiles of perturbations to determine common effects of different perturbations.

54. The method of claim 50, further comprising:
after the step of determining the representative profile, predicting effects of a perturbation from other perturbations with known effects with the most similar representative profiles.
